(19) United States
(12) Patent Application Publication
(10) Pub. No.: US 2010/0248341 A1
(43) Pub. Date: Sep. 30, 2010

(54) TIP DRIVE APPARATUS

(75) Inventors: Yasuo Sasaki, Machida-shi (JP); Yuka Imaoka, Hino-shi (JP); Kiyohiko Tateyama, Sagamihara-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/813,038

(22) Filed: Jun. 10, 2010

Related U.S. Application Data
(63) Continuation of application No. PCT/JP2008/072196, filed on Dec. 5, 2008.

(30) Foreign Application Priority Data
Dec. 10, 2007 (JP) 2007-318891

(51) Int. Cl. C12M 1/00

(52) U.S. Cl. 435/285.1

(57) ABSTRACT

A tip drive apparatus includes a shaft to which a support unit on which a tip unit is formed, and a main unit configured to move the shaft, thereby to move the tip unit toward an object while remaining held at a prescribed angle. The shaft is arranged to meet four requirements including a first requirement of being prevented from contacting a condenser lens of an inverted microscope to which the main unit is attached, a second requirement of providing a region in which a distal end of the support unit is recognized, a third requirement of being prevented from contacting a sidewall of a dish for holding the object, and a fourth requirement of providing a region in which the dish moves relative to the tip unit.

### CROSS REFERENCE TO RELATED APPLICATIONS

This is a Continuation Application of PCT Application No. PCT/JP2008/072196, filed Dec. 5, 2008, which was published under PCT Article 21(2) in Japanese.

This application is based upon and claims the benefit of priority from prior Japanese Patent Application No. 2007-318891, filed Dec. 10, 2007, the entire contents of which are incorporated herein by reference.

### BACKGROUND OF THE INVENTION

1. Field of the Invention

The present invention relates to a tip drive apparatus that can move a tip unit formed on a support unit having flexibility and arranged at a prescribed angle to an object, while holding the tip unit at the prescribed angle.

2. Description of the Related Art

WO 04/092369 discloses a microinjection method and apparatus that are designed to introduce a substance, such as genes, into cell. In the technique disclosed, the substance is electrically adsorbed to the distal end of a microneedle, which is a tip unit, and the microneedle is then inserted into a cell. A pulse voltage is applied to the microneedle, moving the substance from the distal end of the microneedle and introducing the substance into the cell. The microneedle is thrust into the cell as it is minutely moved by using a piezoelectric element that can expand and contract coaxially with the microneedle. This technique is to hold genes at the distal end of the microneedle, can introduce the genes into the cell in a low-invasive manner, increasing the survival rate of the cell.

However, the microneedle cannot penetrate the cell membrane in some cases. This is because the microneedle invades the cell at a very small volume and also because the cell membrane has fluidity. Even if the microneedle is moved to such a position where its distal end may penetrate the cell membrane, the cell membrane covers up the surface of the distal end, disabling the needle from piercing the cell membrane in some cases. Consequently, the tip cannot be stably driven and the substance cannot be introduced at a sufficient rate.

The technique disclosed in the above-identified document cannot utilize a tip drive apparatus that supplies an electric current to the microneedle in order to give an electrical stimulus to a living cell so that the cell may be observed in a living state with high efficiency.

This invention has been made in view of the foregoing. An object of the invention is to provide an apparatus for driving a tip, which can introduce a substance into a cell at in a low-invasive manner, thus maintaining the cell at a high survival rate or apply an electrical stimulus to the cell in order to observe the living cell with high efficiency.

### BRIEF SUMMARY OF THE INVENTION

According to an aspect of embodiments, there is provided a tip drive apparatus capable to moving a tip unit toward an object, while holding the tip unit at a prescribed angle, the tip unit being formed on a support unit having flexibility and directed to the object at the prescribed angle, the apparatus comprising:

a shaft to which the support unit on which the tip unit is formed; and

a main unit configured to move the shaft, thereby to move the tip unit toward the object while remaining held at the prescribed angle,

wherein the shaft is arranged to meet four requirements including:

a first requirement of being prevented from contacting a condenser lens of an inverted microscope to which the main unit is attached;

a second requirement of providing a region in which a distal end of the support unit is recognized;

a third requirement of being prevented from contacting a sidewall of a dish for holding the object; and

a fourth requirement of providing a region in which the dish moves relative to the tip unit.

According to an aspect of embodiments, there is provided a tip drive apparatus capable to moving a tip unit toward an object, while holding the tip unit at a prescribed angle, the tip unit being formed on a support unit having flexibility and directed to the object at the prescribed angle, the apparatus comprising:

a shaft to which the support unit on which the tip unit is formed; and

a main unit configured to move the shaft, thereby to move the tip unit toward the object while remaining held at the prescribed angle,

wherein the shaft is arranged to meet three requirements including:

a first requirement of being positioned outside a region where a condenser lens of an inverted microscope to which the main unit is attached is located, while remaining at a level above a lower surface of the condenser lens;

a second requirement of being positioned outside a region where a distal end of the support unit is recognized, while remaining at a level between the lower surface of the condenser lens and an upper edge of a sidewall of a dish for holding the object; and

a third requirement of being positioned outside the region where the distal end of the support unit is recognized and a region that the sidewall of the dish sweeps as the dish moves outwards relative to the tip unit, while remaining at a level below the upper edge of the sidewall of the dish.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing the overall configuration of a tip drive apparatus according to a first embodiment of this invention.

FIG. 2 is a diagram showing the characterizing section of the tip drive apparatus according to the first embodiment.

FIG. 3 is a diagram showing the configuration of a needle.

FIG. 4 is a diagram explaining a region in which the shaft can be arranged in the tip drive apparatus according to the first embodiment.

FIG. 5 is a sectional view taken along line A-A shown in FIG. 4.

FIG. 6 is a sectional view taken along line B-B shown in FIG. 4.

FIG. 7 is a sectional view taken along line C-C shown in FIG. 4.

FIG. 8 is a diagram explaining the angles the shaft may have if the shaft extends straight.

FIG. 9 is a block diagram showing the electrical configuration of the tip drive apparatus according to the first embodiment.

FIG. 10 is a flowchart explaining a tip driving method using the tip drive apparatus according to the first embodiment.

FIG. 11 is a diagram presenting a microscope image of HelaS3 cells into which tip drive apparatus according to the first embodiment has introduced genes to express GFP fluorescent protein.

FIG. 12 is a diagram presenting an image of the HelaS3 cells, acquired through a phase contrast microscope, 24 hours after the genes had been introduced into the cell to express GFP fluorescent protein.

FIG. 13 is a diagram presenting a microscope image of the HelaS3 cells, acquired through a fluorescence observation, 24 hours after the genes had been introduced into the cell to express GFP fluorescent protein.

FIG. 14 is a diagram showing a characterizing feature for explaining a shape of a shaft in a tip drive apparatus according to a second embodiment of this invention.

FIG. 15 is a diagram showing a modified shaft according to the second embodiment.

FIG. 16 is a diagram showing a characterizing feature for explaining a shaft in a tip drive apparatus according to a third embodiment of this invention.

FIG. 17 is a diagram showing a modified shaft according to the third embodiment.

FIG. 18A is a side view showing a distal end of a needle of a tip drive apparatus according to a fourth embodiment of this invention.

FIG. 18B is a plan view showing the distal end of the needle according to the fourth embodiment.

### DETAILED DESCRIPTION OF THE INVENTION

Some of the best modes for carrying out this invention will be described, with reference to the accompanying drawings.

### First Embodiment

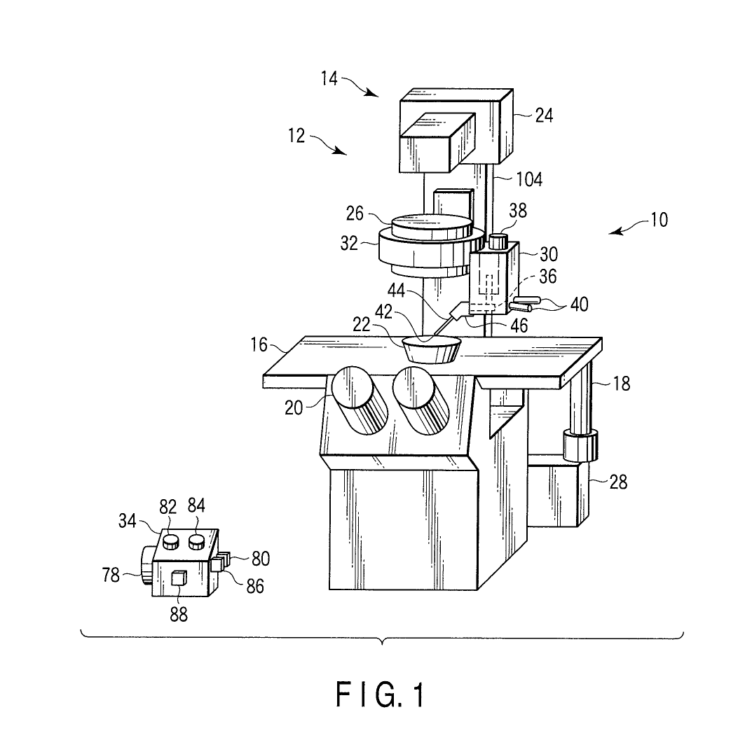

As shown in FIG. 1, a tip drive apparatus 10 according to a first embodiment of this invention is attached to an inverted microscope 12 through which to observe cells. So attached, the tip drive apparatus 10 is used.

The inverted microscope 12 has an illumination device 14, a microscope XY stage 16, a microscope XY stage handle 18, an objective lens (not shown), and an eyepiece 20. The illumination device 14 illuminates the cells set in on a dish 22. The microscope XY stage 16 moves the dish 22 in X direction and Y direction. When operated, the microscope XY stage handle 18 drives the microscope XY stage 16. The objective lens and the eyepiece 20 constitute an optical system through which to observe the light reflected from, or passing through, the cells mounted on the dish 22, or the fluorescent light emanating from the cells. At least the bottom of the dish 22 is made of transparent material such as glass, so that the cells may be observed.

The inverted microscope 12, which is a manually operable type, may be replaced by an electrically-driven type, in which the XY stage 16 is driven and controlled by a computer. Further, the inverted microscope 12 may instead be a type that has a CCD camera and can display images on a monitor.

The illumination device 14 has an illumination light source 24, a condenser lens 26, and an epi-illumination light source 28. The illumination light source 24 applies illumination light to the cells mounted on the dish 22, from the side opposite to the eyepiece 20. The condenser lens 26 receives the illumination light emitted from the illumination light source 24 and converges the light onto the cells. The epi-illumination light source 28 applies illumination light to the cells mounted on the dish 22, from the same side as the eyepiece 20.

The tip drive apparatus 10 according to this embodiment is composed of a main unit 30, a microscope adaptor 32, and an operation module 34. The microscope adaptor 32 is a unit that is attached to the condenser lens 26 of the main unit 30. As seen from FIG. 1, the main unit 30 is attached at the right side of the condenser lens 26, in front of the inverted microscope 12, where the eyepiece 20 is arranged. The operation module 34 is connected by a cable (not shown) to the main unit 30 and can be set at any desired position.

The main unit 30 has an adaptor holder unit 36, a Z-drive unit 38, and a needle-tip XY adjustment knob 40. A tip unit 42, which should be driven, is secured to a needle 44. The needle 44, which has the tip unit 42, is attached to the adaptor 46. The adaptor 46 holding the needle 44 is attached to the adaptor holder unit 36. As the adaptor holder unit 36 is moved in the Z-direction, the Z-drive unit 38 drives the tip unit 42 in the Z-direction. The needle-tip XY adjustment knob 40 moves the adaptor holder unit 36 in X direction and Y direction, adjusting the XY position of the tip unit 42.

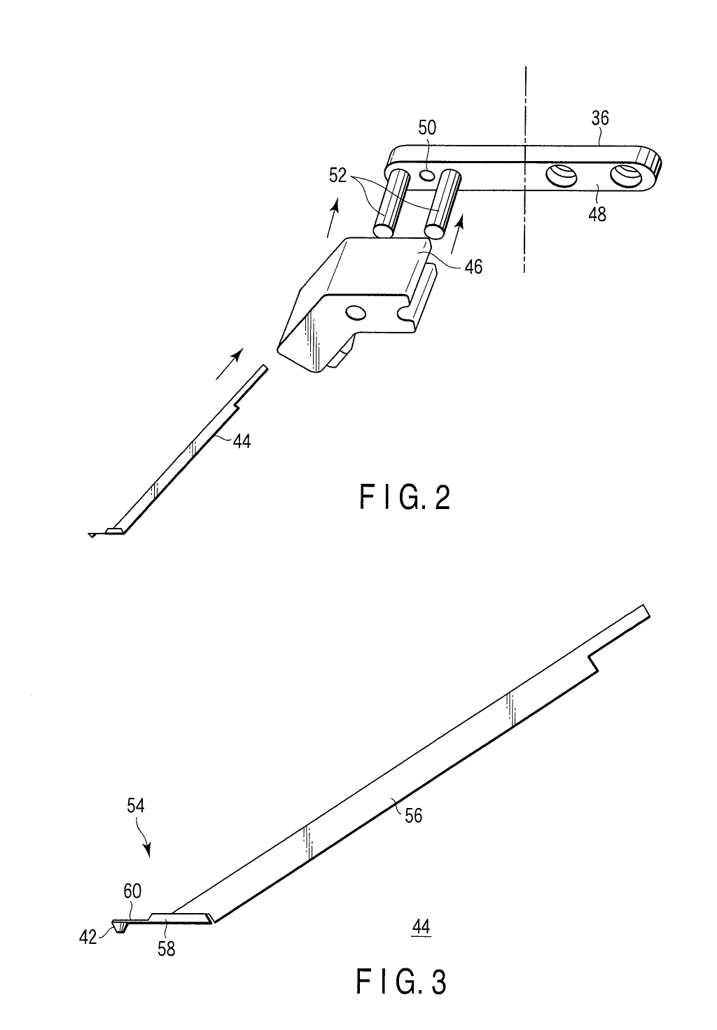

As shown in FIG. 2, the adaptor holder unit 36 has a Z-axis drive holder unit 48 configured to secure it to a linear movement mechanism (not shown) of the Z-drive unit 38 through a XY drive mechanism (not shown) (the needle-tip XY adjustment knob 40 drives the adaptor holder unit 36, in cooperation with the drive mechanism). Moreover, the adaptor holder unit 36 has an attachment member on the side opposite to the Z-axis drive holder unit 48 in the lengthwise direction. The attachment member is configured to attach the adaptor 46 to, and detached the same from, the adaptor holder unit 36. The attachment member is a magnet 50 if the adaptor 46 is made of metal or has a metal component. That section of the adaptor holder unit 36, which is illustrated on the right of the one-dot, dashed line shown in FIG. 2, is incorporated in the main unit 30. That is, the magnet 50 is provided outside the main unit 30. Near the magnet 50, fitting members 52 are provided, and can fit into the holes or grooves made in the adaptor 46 to set the adaptor 46 at a desired position. The fitting members 52 protrude toward the front of the inverted microscope 12. Therefore, the adaptor 46 is attached to the adaptor holder unit 36 by inserting into the holes or grooves from the front of the inverted microscope 12.

Another magnet 50 and other fitting members 52 may be provided on the back of the adaptor holder unit 36 so that the adaptor 46 may be attached to the adaptor holder unit 36 when the main unit 30 is secured to the left side of the condenser lens 26. Alternatively, the adaptor holder unit 36 may be replaced by another, depending upon the position at which the main unit 30 is secured.

As shown in FIG. 3, the needle 44 attached to the adaptor 46 is composed of a cantilever tip 54 and a shaft 56 holding the cantilever tip 54. The cantilever tip 54 has the tip unit 42 mentioned above. The cantilever tip 54 is adhered to the distal end of the shaft 56.

The cantilever tip 54 has been manufactured by a silicon process and is composed of a silicon base unit 58, a flexible lever unit 60, and the above-mentioned tip unit 42. The silicon base unit 58 is a part to which another part, i.e., the shaft 56 is adhered. The lever unit 60 extends from the silicon base unit 58 and has, for example, thickness of 2.7 μm, length of 240 μm and elastic constant of about 2 N/m. The tip unit 42 is formed at the free end of the lever unit 60, at an angle of about 90° to the lengthwise direction of the lever unit 60.

In the tip drive apparatus 10 according to this embodiment, the needle 44 is inserted into, and held in, the hole (not shown) made in the adaptor 46. Thereafter, the adaptor 46, now holding the needle 44, is attached to the main unit 30. The needle 44, which is basically an expendable article and replaced frequently, can thus be replaced by a new one. Therefore, the tip drive apparatus 10 can be used over again, without the risk of contamination.

Assume that the needle 44, which is a thin and long member, is directly attached to the main unit 30. Then, the operability decreases, and the tip unit 42 may hit a part, such as microscope XY stage 16, of the inverted microscope 12 during the attaching operation, and may possibly be broken while being attached to the main unit 30. In this embodiment, the needle 44 is first attached to the adaptor 46 removed from the main unit 30, and the adaptor 46 is then secured at the front of the main unit 30. Hence, the risk of damaging the tip unit 42 can be reduced.

The adaptor 46 is configured to hold the shaft 56 of the needle 44, at a prescribed angle and in a downwardly inclined position, when the adapter is attached to the main unit 30. Further, the cantilever tip 54 is adhered to the shaft 56, at a prescribed angle to the shaft 56. Moreover, as pointed out above, the tip unit 42 is provided, extending in a direction to intersecting with the lengthwise direction of the lever unit 60. The tip unit 42 is therefore held with its distal end directed downward, almost in the vertical direction, at the free end of the lever unit 60, as long as the adaptor 46 remains secured to the main unit 30.

The region, in which the adaptor 46 can be arranged, while holding the shaft 56, will be explained.

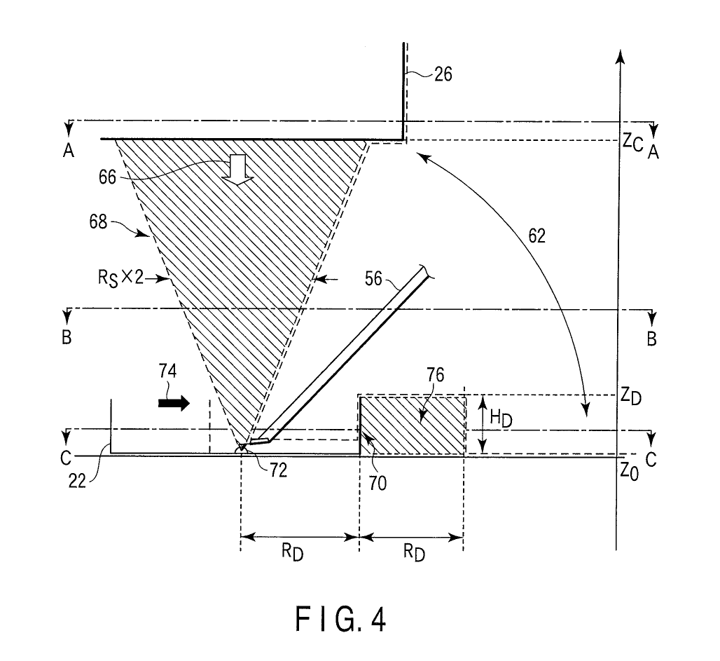

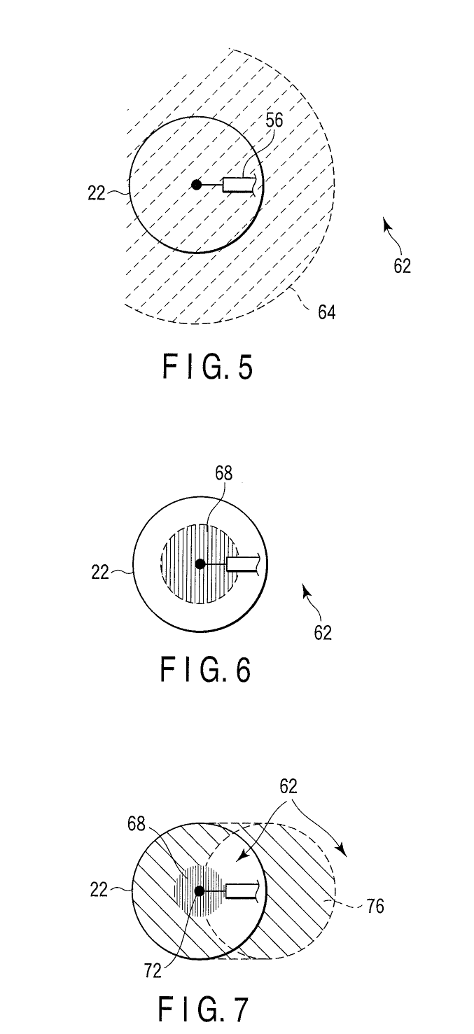

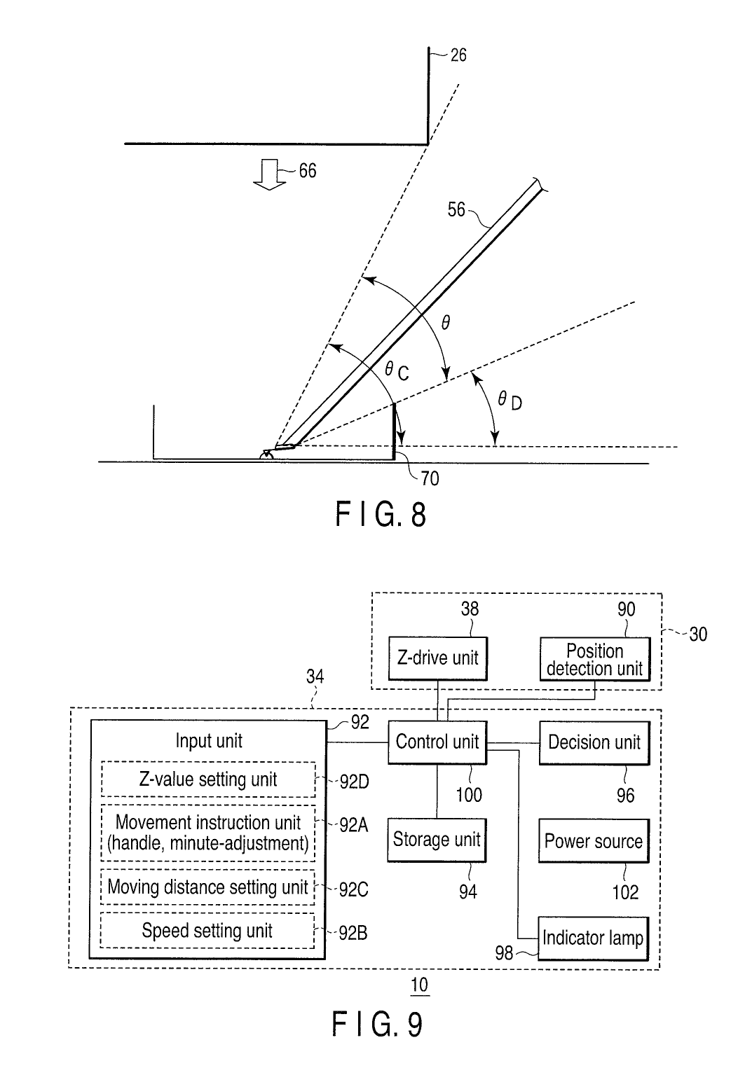

If the adaptor 46 rotates the shaft 56 upwards too much in order to hold the shaft 46 at a fixed angle, it will inevitably interfere with the condenser lens 26. Therefore, the shaft arrangement region 62 should be a region where the shaft 56 is prevented from contacting the condenser lens 26 as shown in FIG. 4 (Requirement 1). This means that the region should not be a region in which the condenser lens 26 is located, above a level Z_{C} that corresponds to the lower side of the condenser lens 26. That is, the shaft arrangement region 62 lies on the side of the main unit 30 with respect to the condenser lens boundary 64, evading the region indicated by hatching in FIG. 5. The condenser lens 26 can be selected from various condenser lenses having different working distances (WDs) and different outside diameters, in accordance with the use. It may be, for example, a condenser lens of ultra long working distance (WD: about 45 mm; outside diameter: about 56 mm at the lowermost surface).

Further, a region must be provided to make it possible to recognize the distal end of the lever 60 (Requirement 2). This is because the shaft arrangement region 62 must not be a region where the shaft 56 blocks the illumination light emitted emerging from the condenser lens 26. The “region where the shaft 56 blocks the illumination light emitted emerging from the condenser lens 26” is one in which the NA of the condenser lens 26 should not decrease much because of the existence of the shaft 56. In other words, the shaft 56 need not be completely outside this region. Hence, in the space below the region Z_{C} corresponding to the lower side of the condenser lens 26 (or the space extending from the region Z_{C} to a region Z₀ corresponding to the bottom of the dish 22), such a lever recognition region 68 indicated by hatching in FIG. 4 should be evaded. That is, the shaft arrangement region 62 is a region excluding the lever recognition region 68 indicated by hatching in FIG. 6.

If the adaptor 46 holds the shaft 56 at an insufficient angle, the shaft 56 will inevitably interfere with the sidewall 70 of the dish. In view of this, the shaft arrangement region 62 must be one where the shaft 56 would not contact the sidewall 70 of the dish (Requirement 3).

Moreover, it is desired that the tip unit 42 should be able to access almost all cells in the dish 22, not only the cells 72 located at the center (initial position) of the dish 22. A region should therefore be provided, which relatively moves as is indicated by the block-line arrow shown in FIG. 4 (Requirement 4). In the case of this embodiment, the tip unit 42 can move relative to the dish 22, by the radius R_{D} of the dish 22, from the center of the dish 22 in the direction opposite to the main unit 30. This is because any region on the bottom of the dish 22 can be accessed as the dish 22 is rotated as needed.

To meet Requirements 3 and 4, the shaft arrangement region 62 is outside a dish movable region 76 indicated by hatching in FIG. 4, in which the dish sidewall 70 sweeps as the dish 22 moves outwards relative to the tip unit 42. The dish movable region 76 is a region determined by the radius R_{D} of the dish 22 and the height H_{D} of the dish sidewall 70. That is, the shaft arrangement region 62 must be outside the dish movable region 76, in the space extending from a region Z_{D} equivalent to the height H_{D} of the dish sidewall 70 (or the space extending from Z_{C} to the region Z₀ corresponding to the bottom of the dish 22). Hence, the shaft arrangement region 62 excludes the lever recognition region 68 and the dish movable region 76, both indicated by hatching in FIG. 7.

As described above, the cantilever tip 54 is secured to the distal end of the shaft 56. Therefore, the shaft arrangement region 62 is, needless to say, narrower than the region Z₀ equivalent to the bottom of the dish 22, by the height of the cantilever tip 54, in the dish 22.

The shaft 56 must be arranged in the shaft arrangement region 62 that meets Requirements 1 to 4 mentioned above.

The shaft 56 may be straight, extending from the adaptor 46 to the tip unit 42. If this is the case, the shaft angle θ has a value between the maximum angle θ_{C} determined by the position and size of the condenser lens 26 and the minimum angle θ_{D} determined by the radium R_{D} of the dish 22 and the height H_{D} of the dish sidewall 70. Assume that the shaft 56 is about 50 mm long, that the dish 22 is a 35 mm glass-bottomed dish generally used in cell culture (radius R_{D}: about 17.5 mm, sidewall height H_{D}: about 11 mm). Then, the maximum angle θ_{C} is about 60°, and the minimum angle is about 30°. In this case, it is desirable to set the shaft angle to 45°, which is the intermediate value in the range of 30° to 60°. If the adaptor 46 holds the shaft 56 at the angle of 45°, the shaft 56 will never interfere with the condenser lens 26 or the dish sidewall 70 during the manipulation performed with respect to the glass surface (diameter: about 14 mm) of the 35 mm glass-bottomed dish. In this case, the dish movable region 76 is a sectional area taken along a line extending from the center of the dish 22 toward the main unit 30 and defined by the radius R_{D} and the sidewall height H_{D}.

Thus, the angle at which the adaptor 46 holds the shaft 56 is set to meet Requirements 1 to 4 mentioned above. The adaptor 46 has a hole (not shown), which extends at this angle and in which the shaft 56 of the needle 44 can be inserted and held fast.

As shown in FIG. 1, the operation module 34 of the tip drive apparatus 10 has a Z-value adjustment handle 78, a speed setting dial 80, a minute-adjustment (up) button 82, a minute-adjustment (down) button 84, a movement setting dial 86, and a Z-value setting button 88.

The Z-value adjustment handle 78 and speed setting dial 80 are used to move coarsely the adaptor holder unit 36 in the Z-direction (in units of millimeters). As the Z-value adjustment handle 78 is rotated, the Z-drive unit 38 drives the adaptor holder unit 36 in the Z-direction, in accordance with the rotation of the Z-value adjustment handle 78. When operated, the speed setting dial 80 switches the distance by which to move the unit 36 as the Z-value adjustment handle 78 is rotated, from any one of the three values, i.e., long, intermediate and short, to another value.

The minute-adjustment buttons 82 and 84 and movement setting dial 86 are used to move minutely the adaptor holder unit 36 in the Z-direction (in units of microns). As the minute-adjustment (up) button 82 or minute-adjustment (down) button 84 is operated, the Z-drive unit 38 drives the adaptor holder unit 36 minutely in the Z-direction, in accordance with the operation of the button. The movement setting dial 86 switches the distance by which to move the unit 36 as the minute-adjustment button 82 or 84 is operated one time, from any one of the three values, i.e., long, intermediate and short, to another value.

The Z-value setting button 88 is a button, which may be pushed to instruct that any position in the Z-direction be stored. Even if the Z-value adjustment handle 78 or the minute-adjustment button 82 or 84 is operated, the adaptor holder unit 36 will never move down below the position stored by pushing the Z-value setting button 88 (toward the sample placed in the dish 22). The Z-value setting button 88 has a latch mechanism (not shown). Once depressed or turned on by the operator, the Z-value setting button 88 remains in the on state until it is depressed again. Hereinafter, the operation of the Z-value adjustment handle 78 and the minute-adjustment buttons 82 and 84 while the Z-value setting button 88 remains in the off state is called the “first mode,” and the operation of the Z-value adjustment handle 78 and the minute-adjustment buttons 82 and 84 while the Z-value setting button 88 remains in the on state is called the “second mode.”

As shown in FIG. 9 illustrating the electrical configuration of the tip drive apparatus 10 according to this embodiment, the main unit 30 has, in addition to the Z-drive unit 38, a position detection unit 90 that is configured to detect the position of the adaptor holder unit 36. The position detection unit 90 may directly detect the position of the adaptor holder unit 36 by using optical means, or may indirectly detect the position of the adaptor holder unit 36 by detecting the distance by which the Z-drive unit 38 has been driven. Furthermore, the position detection unit 90 may be provided as a unit separated from the main unit 30.

The operation module 34 has an input unit 92, a storage unit 94, a decision unit 96, an indicator lamp 98, a control unit 100, and a power source 102.

The input unit 92 includes a movement instruction unit 92A, a speed setting unit 92B, a moving distance setting unit 92C, and a Z-value setting unit 92D. The movement instruction unit 92A outputs a movement instruction when the Z-value adjustment handle 78 is operated and the minute-adjustment button 82 or 84 is turned on. The speed setting unit 92B outputs a speed setting signal representing the moving speed set as the speed setting dial 80 is rotated. The moving distance setting unit 92C outputs a distance setting signal representing the distance set as the movement setting dial 86 is rotated. The Z-value setting unit 92D outputs a Z-value setting signal when the Z-value setting button 88 is turned on. The signals output from the input unit 92 are input to the control unit 100.

The storage unit 94 stores, as the Z-value, the position of the adaptor holder unit 36, which the position detection unit 90 detects when the Z-value setting button 88 is turned on. The decision unit 96 compares the position of the adaptor holder unit 36, which the position detection unit 90 has detected, with the Z-value stored in the storage unit 94, thereby determining whether the adaptor holder unit 36 has reached the position of the Z-value. The indicator lamp 98 blinks in response to the Z-value setting signal output from the Z-value setting unit 92D. Seeing the indicator lamp 98 blinking, the operator can confirm that the Z-value has been duly stored.

The control unit 100 controls the entire tip drive apparatus 10. The power source 102 supplies power to the components of the tip drive apparatus 10.

A tip driving method, which uses the tip drive apparatus 10 according to this embodiment, will be explained below.

Here, a case will be described, in which the tip drive apparatus 10 according to this embodiment is used to introduce a substance into cells being cultured in a culture solution filled in the dish 22.

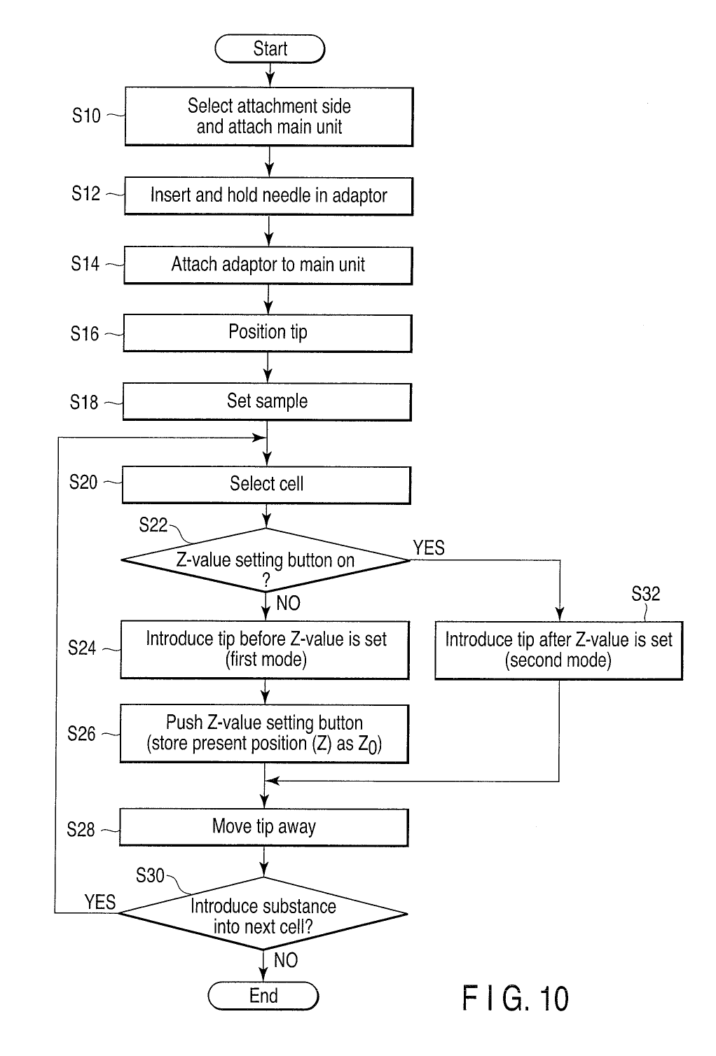

As shown in FIG. 10, the side to which the main unit 30 should be attached is selected, and the main unit 30 is then attached to the condenser lens 26 via the microscope adaptor 32 (Step S10).

Next, the needle 44 is inserted into, and held in, the adaptor 46 removed from the main unit 30 (Step S12). The adaptor 46 holding the needle 44 is attached to the adaptor holder unit 36 of the main unit 30, from the front of the inverted microscope 12 (Step S14).

Thereafter, the tip is positioned (Step S16). That is, while observing the needle 44, the operator brings the tip unit 42 formed at the distal end of the needle, to the center (i.e., view-field center) of the eyepiece (not shown). The operator accomplishes this by manipulating the needle-tip XY adjustment knob 40 of the main unit 30 and the Z-value adjustment handle 78 of the operation module 34. This manipulation is performed, not having the dish 22 mounted on the microscope XY stage 16. As for the Z-direction, the operator turns the speed setting dial 80 of the operation module 34, setting the long or intermediate distance, and then operates the Z-value adjustment handle 78, thereby lowering the tip unit 42 to a position where he or she can see the lever unit 60 of the cantilever tip 54.

When the tip unit 42 is so positioned, a sample is set, more precisely, the dish 22 is mounted on the microscope XY stage 16 (Step S18). This step is performed in the following sequence. First, the Z-value adjustment handle 78 of the operation module 34 is operated, moving the tip unit 42 at the distal end of the needle 44, to a safe region (upward in the Z-direction). An arm 104 (FIG. 1) of the inverted microscope 12 is then pulled back. As a result, the main unit 30 is moved as a whole. A space for sample setting is thereby provided. Then, the dish 22 (sample) is mounted on the microscope XY stage 16. Thereafter, the arm 104 of the inverted microscope 12 is moved to the initial position. Note that the dish 22 (sample) so set contains a substance in a dispersed state, which will be introduced into cells being cultured in a culture solution held in the dish 22.

Next, the cell into which the substance should be introduced is selected (Step S20). First, the operator manipulates the microscope XY stage handle 18, moving the microscope XY stage 16 and, thereby, arranging the cell held in the dish 22 into the view field of the microscope so that the cell may be observed. Thereafter, the operator actuates the Z-drive unit 38, moving the tip unit 42 of the needle 44 toward the cell from above. That is, while observing through the eyepiece 20, the operator lowers the tip unit 42 in the Z-direction until the lever unit 60 of the cantilever tip 54 comes into the view field and become visually confirmed. This is achieved by first turning the speed setting dial 80 of the operation module 34, setting the low speed, and then operating the Z-value adjustment handle 78. Since the cells in the dish 22 are not at the same height as the tip unit 42, the tip unit 42 is not focused and can hardly be observed. Therefore, the operator moves the tip unit 42 down in the Z-direction, using the lever unit 60 as index. This is because the lever unit 60 is larger than the tip unit 42 and therefore the lever unit 60 can be recognized generally, even if its image is not focused. After the lever unit 60 moves to a position where it is visually recognized, the operator adjust the position of the microscope XY stage 16 with respect to X direction and Y direction, while observing the cells through the eyepiece 20. The tip unit 42 is thereby set at a position that seems right above the cell into which to introduce the substance. Thus, the cell into which to introduce the substance is selected.

The following operation depends on whether the Z-value has been set or not in the storage unit 94 of the operation module 34.

When the tip is driven for the first time, the Z-value has yet to be set in the storage unit 94 (Step S22). Therefore, the tip is introduced in the first mode (without using the Z-value) (Step S24). That is, the operator determines an optimal position in the Z-direction, while operating the Z-value adjustment handle 78 or the minute-adjustment button 82 or 84 and observing through the eyepiece 20, confirming “the distortion of the cell” or “the bending of the lever unit 60.” At this point, the Z-value adjustment handle 78 is manipulated, while the speed setting dial 80 is turned, switching the speed from any one of the three values, i.e., high, medium and low, to another value. The minute-adjustment button 82 or button 84 is operated, while the movement setting dial 86 is turned, switching the distance from any one of the three values, i.e., long, intermediate and short, to another value.

As the tip unit 42 is thus moved down toward the bottom of the dish 22, lowering the distal end of the tip unit 42, the tip unit 42 contacts the cell in the dish 22. If the tip unit 42 is further lowered, the distal end of the tip unit 42 will pass through the cell membrane and penetrates the cell nucleus, forming a scar or hole in the membrane and nucleus. The substance dispersed in the dish 22 therefore flows into the cell through the formed scar or hole. The substance may flow into the cell, without forming a scar or hole, depending on the size of particles to introduce, if the channel coupled to a stretch receptor or the like is opened when the tip unit 42 deforms the cell, applying a physical stimulus to the cell. Thus, the substance is introduced.

When the substance is so introduced, the operator may push the Z-value setting button 88 of the operation module 34. Then, the control unit 100 of the operation module 34 determines that the Z-value setting button 88 has been pushed. In this case, the control unit 100 makes the storage unit 94 stores, as the Z-value representing an optimal position, the present position of the adaptor holder unit 36, which the position detection unit 90 has detected (Step S26). At this point, the control unit 100 turns on the indicator lamp 98.

Thereafter, the operator operates the Z-value adjustment handle 78 of the operation module 34, raising the needle 44 and, thus, moving up the tip unit 42 (Step S28). That is, the operator turns the speed setting dial 80 of the operation module 34, switching the speed to the medium speed or the low speed, and then operates the Z-value adjustment handle 78, raising the tip unit 42.

After the tip unit 42 is raised and pulled from the cell, and after a certain time has passed, the cell membrane is restored by itself and now contains the substance.

Assume that the tip unit 42 has been completely moved up (Step S28). Then, whether the substance should be introduced into the next sample cell is determined (Step S30). If NO, the operator turns off the power switch (not shown) of the main unit 30, terminating the tip driving method.

On the other hand, if the substance should be introduced into any other cell (Step S30), the method returns to Step S20. Thus, the substance may be introduced into other sample cells, one after another. That is, the operator manipulates the microscope XY stage handle 18, while making observation through the eyepiece 20, thereby actuating the microscope XY stage 16 and setting the tip unit 42 right above the cell into which to introduce the substance. In other words, the operator selects the cell into which the substance should be introduced (Step S20).

At the time the tip is driven for the second time, and so forth, the Z-value has already been set in the storage unit 94 (Step S22). Therefore, the tip is introduced in the second mode (by using the Z-value) (Step S32). In this case, the Z-value has been set in the storage unit 94. Therefore, the operator can lower the tip unit 42 to an optimal position, merely by fully lowering the tip unit 42, without worrying about an excessive manipulation of the Z-value adjustment handle 78 and minute-adjustment buttons 82 and 84, once the tip unit 42 has been positioned in the horizontal direction. That is, the decision unit 96 of the operation module 34 compares the position of the adaptor holder unit 36, detected by the position detection unit 90, with the Z-value set in the storage unit 94, determining whether the adaptor holder unit 36 (tip unit 42) has reached the position of the Z-value. If the decision unit 96 determines that the adaptor holder unit 36 is found to have reached this position, the control unit 100 of the operation module 34 controls the Z-drive unit 38, preventing the same from moving down further, even if the Z-value adjustment handle 78 and the minute-adjustment buttons 82 and 84 are operated.

Since the optimal Z-position is set in the storage unit 94, the adaptor holder unit 36 (tip unit 42) may be automatically lowered to the optimal Z-position. That is, the handle operation in the second mode may be automated.

If the inverted microscope 12 is an electrically-driven type, a computer controls and drives the microscope XY stage 16. In this case, the inverted microscope 12 has a CCD camera or the like, and can display images on a monitor. If the inverted microscope 12 is such an electrically-driven type, not a manually operable type, any cell into which the substance should be introduced may be selected on the monitor screen, and the tip unit may be automatically moved to its position. In other words, the microscope XY stage 16 may be automatically adjusted in the XY direction.

Note that the substance to introduce into the cell may be anything that can be dispersed in the dish 22, such as genes, dyes, fluorescent reagent, e.g., quantum dots, ions, peptides, proteins or polysaccharides.

### EXAMPLES

A first example is HelaS3 cells which were immersed in a gene solution, and into which genes were introduced. The genes express GFP fluorescent protein. Whether the genes were successfully introduced or not can be confirmed by performing fluorescence observation.

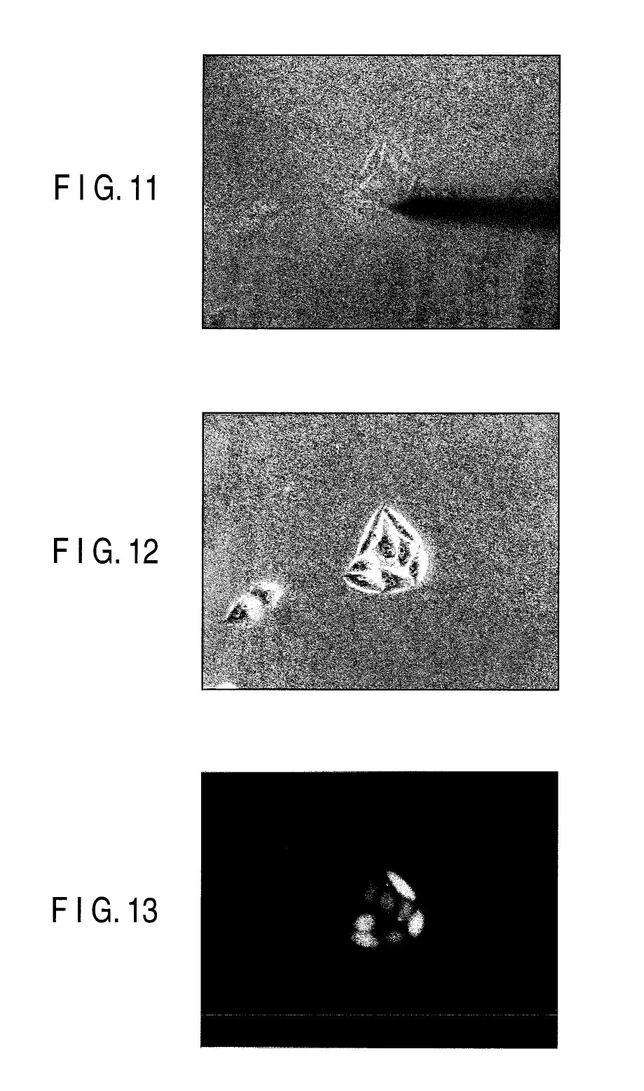

FIG. 11 presents a microscope image of the cells, obtained immediately after genes had been introduced. FIG. 12 and FIG. 13 are microscope images of the cells, acquired 24 hours after introducing the genes, which show whether the genes have been introduced into the cells. The image of FIG. 12 is one observed through a phase contrast microscope, showing the state the cells had 24 hours after the genes had been introduced. FIG. 13 is one obtained through fluorescence observation. In the cell into which the genes had been successfully introduced, the genes well expressed, attaining intense fluorescent light. This proves that the genes were introduced into the cells at a very high efficiency.

The tip drive apparatus 10 according to this embodiment can therefore access many cells placed on the dish 22. The apparatus 10 can therefore introduce a substance into any cell not only in such a low-invasive manner while maintaining such a high cellular survival rate as the conventional tip drive apparatus, but also with high reliability and high efficiency.

Further, the tip drive apparatus 10 according to this embodiment can move the tip unit 42 and the dish 22 relative to each other. The apparatus 10 can therefore cover the entire region of the dish 22.

### Second Embodiment

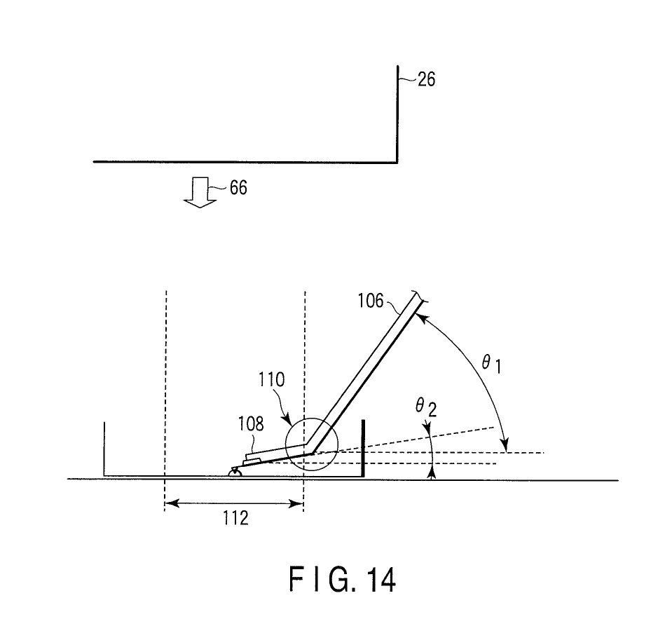

In the first embodiment, a straight shaft 56 is used, which extends from the adaptor 46 to the tip unit 42. The tip drive apparatus according to a second embodiment of this invention uses such a non-straight shaft 56 as shown in FIG. 14. The non-straight shaft 56 has a first shaft part 106, a second shaft part 108, and a connection part 110. The first haft part 106 is located at the adaptor 46. The second shaft part 108 is located at the tip unit 42. The connection part 110 is bent at least once, and connects the first shaft part 106 and the second shaft part 108.

In this case, the first angle θ₁, which the first shaft part 106 extends, to the XY plane (i.e., the surface on which the dish 22 lies) should be larger than the second angle θ₂, which the second shaft part 108 extends, to the XY plane. Moreover, it is desired that the first shaft part 106 should lie outside the effective illumination region 112 that contributes to the illumination of the cells 72.

The tip drive apparatus according to the second embodiment, so configured as described above, can introduce a substance into any cell not only in such a low-invasive manner while maintaining such a high cellular survival rate as the conventional tip drive apparatus, but also with high reliability and high efficiency, as does the tip drive apparatus according to the first embodiment.

Further, since the first shaft part 106 lies outside the effective illumination region 112, the first shaft part 106 would not block the illumination light 66 emerging from the condenser lens 26. That is, the closer to the condenser lens 26 the first shaft part 106 lies, the larger the blocked amount of illumination light. The farther from the condenser lens 26 the first shaft part 106 is, the smaller the blocked amount of illumination light. Since the first shaft part 106 which is close to the condenser lens 26 lies outside the effective illumination region 112, the illumination of the cell is influenced but very little.

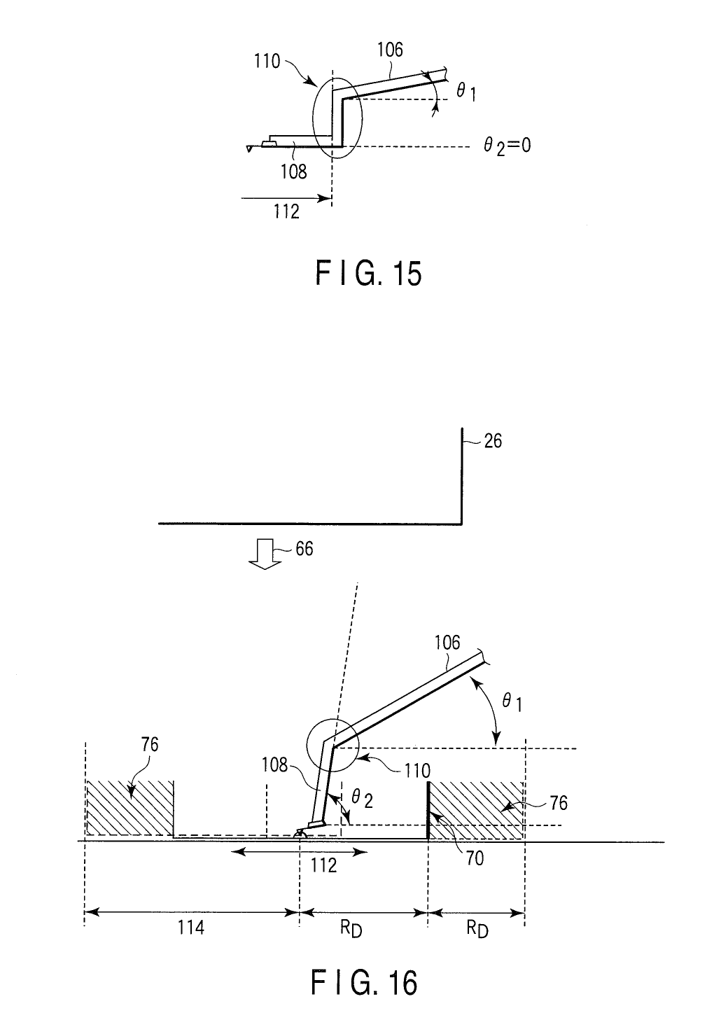

The connection part 110, which connects the first shaft part 106 and the second shaft part 108, is not limited one that is bent only once as shown in FIG. 14. Rather, a connection part that is bent twice as shown in FIG. 15, or bent more times.

### Third Embodiment

As shown in FIG. 16, the tip drive apparatus according to a third embodiment of this invention has a shaft 56, which is bent in the direction opposed to the direction in the second embodiment. That is, the angle θ₁ at which the first shaft part 106 is inclined to the XY plane is smaller than the angle θ₂ at which the second shaft part 108 is inclined to the XY plane. In this case, the second shaft part 108 should preferably be long enough, extending to a level higher than the height H_{D} of the dish sidewall 70.

The tip drive apparatus according to a third embodiment can also introduce a substance into any cell not only in such a low-invasive manner while maintaining such a high cellular survival rate as the conventional tip drive apparatus, but also with high reliability and high efficiency, as does the tip drive apparatus according to the first embodiment described above.

In the present embodiment, the region 114 in which the dish 22 and the shaft 56 move relative to each other has a radius equal to or smaller than 2R_{D}. The access region, in which an access can be made to the bottom of the dish 22 not rotated, therefore expands more than otherwise.

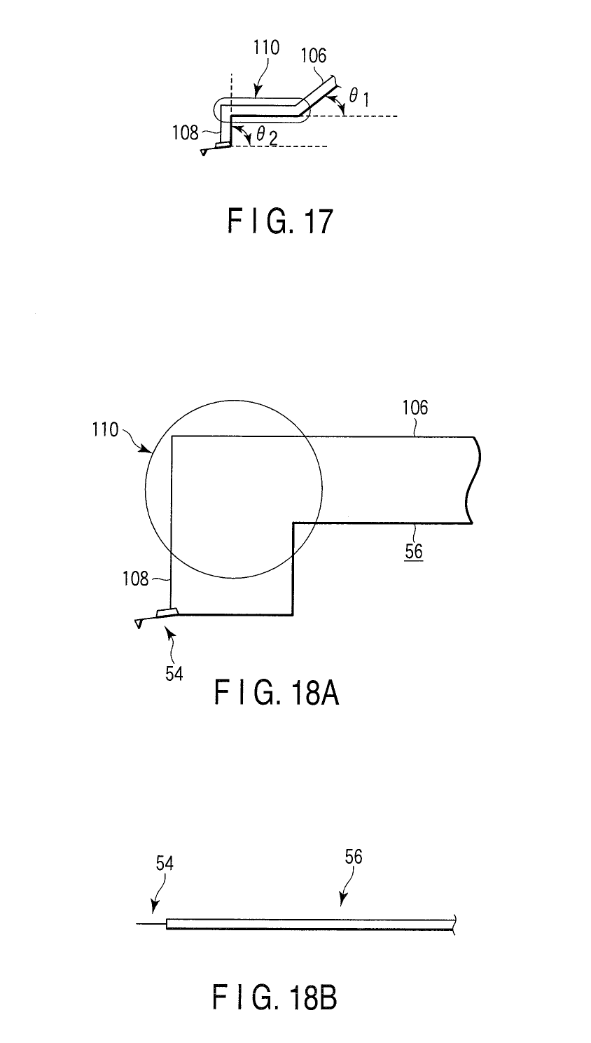

The connection part 110, which connects the first shaft part 106 and the second shaft part 108, is not limited one that is bent only once as shown in FIG. 16. Rather, a connection part that is bent twice as shown in FIG. 17, or bent more times.

### Fourth Embodiment

The illumination light coming from the condenser lens 26 is less blocked if the shaft 56 is a thin rod as in the first to third embodiments. Nonetheless, the shaft 56 not limit to the rod, and may be replaced by such a thin plate as shown in FIGS. 18A and 18B. In this case, too, the same advantages will be achieved as in the first to third embodiments.

### Fifth Embodiment

In the first to fourth embodiments described above, one tip drive apparatus 10 is secured to the inverted microscope 12 and is used. A plurality of tip drive apparatuses 10 may be used at the same time. For example, main units 30 may be secured to the sides of the condenser lens 26.

If a plurality of tip drive apparatuses 10 are used in this manner, tips can be used not only to introduce substances, but also to, for example, apply an electric signal between the tip units 42, thereby to give cells an electrical stimulus. The electrical stimulus is not necessarily be given exclusively by using a plurality of tip units 42. Rather, it can be given by applying a potential difference between a tip unit 42 and a particular electrode (e.g., glass bottom with ITO). If this is the case, the tip unit 42 had better be electrically conductive.

Thus, this embodiment can apply an electrical stimulus to cells, not only in such a low-invasive manner and maintaining the cell at a high survival rate, enabling the operator to observe the living cells with high efficiency.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A tip drive apparatus capable to moving a tip unit toward an object, while holding the tip unit at a prescribed angle, the tip unit being formed on a support unit having flexibility and directed to the object at the prescribed angle, the apparatus comprising:
a shaft to which the support unit on which the tip unit is formed; and
a main unit configured to move the shaft, thereby to move the tip unit toward the object while remaining held at the prescribed angle,
wherein the shaft is arranged to meet four requirements including:
a first requirement of being prevented from contacting a condenser lens of an inverted microscope to which the main unit is attached;
a second requirement of providing a region in which a distal end of the support unit is recognized;
a third requirement of being prevented from contacting a sidewall of a dish for holding the object; and
a fourth requirement of providing a region in which the dish moves relative to the tip unit.

2. The tip drive apparatus according to claim 1, wherein
the shaft includes a first shaft part located at the main unit, a second shaft part located at the tip unit, and a connection part bent at least once and connecting the first shaft part and the second shaft part; and
a first angle at which the first shaft part is inclined to the plane in which the dish is placed is larger than a second angle at which the second shaft part is inclined to the plane in which the dish is placed.

3. The tip drive apparatus according to claim 2, wherein the first shaft part is outside an effective illumination region that contributes to the illumination of a cell.

4. The tip drive apparatus according to claim 1, wherein
the shaft includes a first shaft part located at the main unit, a second shaft part located at the tip unit, and a connection part bent at least once and connecting the first shaft part and the second shaft part; and
a first angle at which the first shaft part is inclined to the plane in which the dish is placed is smaller than a second angle at which the second shaft part is inclined to the plane in which the dish is placed.

5. The tip drive apparatus according to claim 4, wherein a region where the dish moves relative to the tip unit has a radius equal to or smaller than 2R_{D}, where R_{D} is a radius of the dish.

6. The tip drive apparatus according to claim 1, wherein the shaft
extends straight from the main unit to the tip unit and
is arranged at an angle having a value between a maximum angle determined by the position and size of the condenser lens and a minimum angle determined by the radium of the dish and height of the dish sidewall of the dish.

7. A tip drive apparatus capable to moving a tip unit toward an object, while holding the tip unit at a prescribed angle, the tip unit being formed on a support unit having flexibility and directed to the object at the prescribed angle, the apparatus comprising:
a shaft to which the support unit on which the tip unit is formed; and
a main unit configured to move the shaft, thereby to move the tip unit toward the object while remaining held at the prescribed angle,
wherein the shaft is arranged to meet three requirements including:
a first requirement of being positioned outside a region where a condenser lens of an inverted microscope to which the main unit is attached is located, while remaining at a level above a lower surface of the condenser lens;
a second requirement of being positioned outside a region where a distal end of the support unit is recognized, while remaining at a level between the lower surface of the condenser lens and an upper edge of a sidewall of a dish for holding the object; and
a third requirement of being positioned outside the region where the distal end of the support unit is recognized and a region that the sidewall of the dish sweeps as the dish moves outwards relative to the tip unit, while remaining at a level below the upper edge of the sidewall of the dish.

8. The tip drive apparatus according to claim 7, wherein
the shaft includes a first shaft part located at the main unit, a second shaft part located at the tip unit, and a connection part bent at least once and connecting the first shaft part and the second shaft part; and
a first angle at which the first shaft part is inclined to the plane in which the dish is placed is larger than a second angle at which the second shaft part is inclined to the plane in which the dish is placed.

9. The tip drive apparatus according to claim 8, wherein the first shaft part is outside an effective illumination region that contributes to the illumination of a cell.

10. The tip drive apparatus according to claim 7, wherein
the shaft includes a first shaft part located at the main unit, a second shaft part located at the tip unit, and a connection part bent at least once and connecting the first shaft part and the second shaft part; and
a first angle at which the first shaft part is inclined to the plane in which the dish is placed is smaller than a second angle at which the second shaft part is inclined to the plane in which the dish is placed.

11. The tip drive apparatus according to claim 10, wherein a region where the dish moves relative to the tip unit has a radius equal to or smaller than 2R_{D}, where R_{D} is a radius of the dish.

12. The tip drive apparatus according to claim 7, wherein the shaft
extends straight from the main unit to the tip unit and
is arranged at an angle having a value between a maximum angle determined by the position and size of the condenser lens and a minimum angle determined by the radium of the dish and height of the dish sidewall of the dish.

13. The tip drive apparatus according to claim 7, wherein the region that the sidewall of the dish sweeps has a sectional area taken along a line extending from the center of the dish toward the main unit and defined by a radius and a sidewall height of the dish.
